(19) United States
(12) Patent Application Publication
(10) Pub. No.: US 2002/0046002 A1
(43) Pub. Date: Apr. 18, 2002

(54) Method to evaluate the quality of database search results and the performance of database search algorithms

(76) Inventors: Chao Tang, New York, NY (US); Wenzhu Zhang, New York, NY (US); David Fenyo, New York, NY (US); Brian T. Chait, New York, NY (US)

Correspondence Address: Irving N. Feit, Esq., HOFFMANN & BARON, LLP, 6900 Jericho Turnpike, Syosset, NY, 11791, US

(21) Appl. No.: 09/758,027

(22) Filed: Jan. 10, 2001

Related U.S. Application Data
(63) Continuation of application No. 09/591,419, filed on Jun. 10, 2000, now abandoned.

(51) Int. Cl. G01N 33/48; G06F 19/00

(52) U.S. Cl. 702/182; 702/19

(57) ABSTRACT

A method for evaluating the performance of biopolymer identification algorithms, the method comprising a) generating noise and signal distributions, wherein the noise distribution comprises identification results obtained from the search of a database for arbitrarily generated sets of database mass data and wherein the signal distribution comprises identification results obtained from the search of a database for arbitrarily generated sets of database mass data which comprises mass data of a particular biopolymer of the database designated as a signal biopolymer; and b) calculating a performance index from the distributions which evaluates the performance of the algorithm.

### BACKGROUND

Mass spectrometry peptide mapping combined with protein sequence database search is a preferred method for fast protein identification. Several algorithms have been developed for the database search. However, the identification results generated by such algorithms may be based upon a random matching of the unknown protein with a database protein and thereby be inaccurate. It is left to the user to evaluate the results. Thus, the protein identification process using algorithms is a decision making process in which both an identification algorithm's sensitivity and human judgement (a willingness to accept an identification algorithm's result) are key factors. Such factors bring subjectivity into the identification process, greatly diminishing the accuracy of the process.

An objective standard is needed for the evaluation of protein identification process. The present invention provides a method by which to objectively evaluate the reliability/sensitivity of protein identification algorithms. Additionally, the present invention provides an objective method by which to evaluate an individual protein identification result.

### SUMMARY

This and other objects, as will be apparent to those having ordinary skill in the art, have been met by providing a method for evaluating the performance of biopolymer identification algorithms. The method comprises a) generating noise and signal distributions, wherein the noise distribution comprises identification results obtained from the search of a database for arbitrarily generated sets of database mass data and wherein the signal distribution comprises identification results obtained from the search of a database for arbitrarily generated sets of database mass data which comprises mass data of a particular biopolymer of the database designated as a signal biopolymer; and calculating a performance index from the distributions which evaluates the performance of the algorithm.

The invention further comprises a method for evaluating the reliability of a biopolymer identification result. The method comprises generating noise and signal distributions, wherein the noise distribution comprises identification results obtained from the search of a database for arbitrarily generated sets of database mass data and wherein the signal distribution comprises identification results obtained from the search of the database for arbitrarily generated sets of database mass data and mass data of the unknown biopolymer; and calculating a performance index from the distributions which is a function of the reliability of a biopolymer identification result.

### DESCRIPTION OF FIGURES

FIG. 1a shows signal and noise distributions divided by the criterion score for acceptance (“yes”) or rejection (“no”) of identifications which divides the two distributions into four different regions

FIG. 1b shows a “receiver operating characteristics (ROC)” curve plotted for three signal-noise distributions.

FIG. 2 shows the signal-noise distributions and ROC curve for the 25kDa protein using algorithms A-D.

FIG. 3 shows the signal-noise distributions and ROC curve for a 50kDa protein using algorithms A-D.

FIG. 4 shows the signal-noise distributions and ROC curve for a 100kDa using algorithms A-D.

FIG. 5 shows the signal-noise distributions and ROC curve for a 200kDa protein using algorithms A-D.

FIG. 6 shows a) the relative sensitivity (A′/ A′ₘₐₓ) and b) the relative maximum correct hit (Hitₘₐₓ/ max(Hitₘₐₓ)) of three algorithms (A, B, C).

FIG. 7 shows the separation between signal and noise populations (index of separability, d′)

FIG. 8 (a and b) shows the signal and noise populations for 100 experimental data sets and its estimates using the IntelliDuo© algorithm from one set of experimental data.

FIG. 9a shows population estimates for one set of experimental data with six matches out of a total of 43 masses.

FIG. 9b shows an ROC curve in which the hit rate is 75% at a 5% false-alarm rate and the maximum hit rate is 91% (at a false-alarm rate of 100%).

FIG. 9c shows a subset of data with four matches out of total nine masses.

FIG. 10 shows the effect of including partial oxidation on methionine residues. FIG. 10a shows the population estimates from a total of eight experimental masses where four match with the unmodified authentic protein (yeast Nup 170). FIG. 10bshows that an additional match shifted the signal population to a higher score, while the noise population changed very little where partial oxidation is included in the search.

FIG. 11a shows the population estimates for a set of experimental data (from yeast Nup 170) where “all-taxa” is used in database searches.

FIG. 11b shows that the signal population does not change, while the noise population shifts to a lower score, when yeast species information is used in the searches of FIG. 11a.

### DETAILED DESCRIPTION

In one embodiment the invention provides a method for evaluating the performance of biopolymer identification algorithms utilizing signal detection theory. A biopolymer identification algorithm is any algorithm that provides identification results for an unknown biopolymer. Identification results can include a list of biopolymers candidates identified as having a certain probability of being the unknown biopolymer.

A biopolymer is any biological molecule that can be degraded into constituent parts. The degradation is preferably into constituent parts at predictable positions to form predictable masses. Examples of biopolymers include proteins, nucleic acid molecules, polysaccharides and carbohydrates.

Proteins are polymers of amino acids. Constituent parts of proteins comprise amino acids. A protein typically contains approximately at least about ten amino acids, more typically at least about fifty amino acids, and most typically at least about 100 amino acids. In this specification the term proteins include oligopeptides and polypeptides.

Nucleic acids are polymers of nucleotides. Constituent parts of nucleic acids comprise nucleotides. Typically, a nucleic acid contains at least about 100 nucleotides, often at least about 500 nucleotides.

Polysaccharides are polymers of monosaccharides. Constituent parts of polysaccharides comprise one or more monosaccharides. Typically, a polysaccharide contains at least five monosaccharides, more typically at least ten monosaccharides.

Biopolymers for the purposes of this invention include mixtures of biopolymers.

There are various algorithms that attempt to identify unknown proteins. These algorithms use the experimentally obtained data of unknown proteins and compare it with the data of known proteins in a database. A simple algorithm for the measure of similarity calculates the number of experimental masses that are similar to at least one theoretical mass. For example, the masses of an experimental peptide map of an enzymatically digested unknown protein can be compared with the theoretical masses calculated by applying the rules for the specificity of the enzyme to the amino acid sequence of a database protein. These algorithms yield search results which include the protein identified and an identification score on the basis of the similarity of the data of the unknown protein and the data base proteins.

Examples of commercially available protein identification algorithms include Bayesian ProFound, “MOWSE (modified)” (MS-Fit), “MOWSE (probability based)” (Mascot) and “Number of Matches” (MS-Fit). Sophisticated algorithms can be used to generate a score. For example, ProFound (ProteoMetrics) is a software tool for searching protein sequence databases. ProFound's score is “the probability that a specific protein is the protein being analyzed” using a Bayesian statistical framework.

In one embodiment the invention measures the performance of biopolymer identification algorithms. The performance of a biopolymer identification algorithm can be measured by the ability of an algorithm to separate correct biopolymer identifications from other randomly matched biopolymers. Accordingly, performance includes a measure of the sensitivity of an algorithm to differentiate between a biopolymer identification that is correct and an identification that occurs by a random match between a database biopolymer and the unknown biopolymer.

A biopolymer identification is a decision making process based on two key factors: a biopolymer identification algorithm's sensitivity and human judgement. Signal detection theory provides the tools to evaluate the sensitivity of an algorithm objectively by separating these two factors.

Within the framework of signal detection theory, the correct identifications of a biopolymer by an identification algorithm and the random matches of a biopolymer with a database biopolymer constitute two different distributions. Correct identifications are also known as signal; random matches are also known as noise.

The method of the invention for evaluating the performance of biopolymer identification algorithms includes generating signal and noise distributions. The signal distribution includes identification results obtained from the search of a biopolymer database for arbitrarily generated sets of database mass data that includes mass data of a particular biopolymer of the database. This particular biopolymer is designated as the signal protein. The noise distribution includes identification results obtained from the search of a biopolymer database for arbitrarily generated sets of database mass data that may or may not include mass data from the signal protein.

Arbitrary generation of sets means any generation of sets that is based on or determined by individual preference or convenience. These distributions can be generated by any method that arbitrarily generates noise and function distributions for biopolymer identification results.

Mass data of biopolymers are quantifiable information about the masses of the constituent parts of the biopolymer. Mass data include individual mass spectra and groups of mass spectra. The mass spectra can be in the form of peptide maps, oglionucleotide maps or oligosaccharide maps. For the purposes of this invention, mass data include protein mass data of the full length protein or fragments thereof.

For example, mass data for proteins can be generated in any manner which provides protein mass data within a certain accuracy. Examples include matrix-assisted laser desorption/ionization mass spectrometry, electrospray ionization mass spectrometry, chromatography and electrophoresis. Mass data can also be generated by a general purpose computer configured by software or otherwise.

For the purposes of the present invention the mass data, for example a peptide mass, m₁, is determined to an accuracy ±Δm₁, with Δm₁/m₁, preferably <10,000 ppm, more preferably <100ppm and most preferably <30ppm.

A step in generating mass data of a biopolymer can include first cleaving the biopolymer into constituent parts. Biopolymers can be cleaved by methods known in the art. Preferably, the biopolymers are cleaved into constituent parts at predictable positions to form predictable masses. Methods of cleaving include chemical degradation of the biopolymers. Biopolymers can be degraded by contacting the biopolymer with any chemical substance.

For example, proteins can be predictably degraded into peptides by means of cyanogen bromide and enzymes, such as trypsin, endoproteinase Asp-N, V8 protease, endoproteinase Arg-C, etc. Nucleic acids can be predictably degraded into constituent parts by means of restriction endonucleases, such as Eco RI, Sma I, BamH I, Hinc II, etc. Polysaccharides can be degraded into constituent parts by means of enzymes, such as maltase, amylase, alpha-mannosidase, etc.

Mass data of database biopolymers is provided under a particular experimental condition. Experimental conditions are any conditions under which mass data is generated. Examples of experimental conditions include the manner in which cleavage of the biopolymers is accomplished, that is, the specific substance used for the chemical degradation of the biopolymers. Additionally, the experimental condition defines the efficiency of the chemical degradation. The efficiency of a chemical degradation specifies the number of potential cleavage sites that may be expected to remain uncleaved. The mass data generated from the biopolymer database may include mass data representing biopolymers with incomplete cleavages.

A biopolymer database is any compilation of information about characteristics of biopolmers. Databases are the preferred method for storing both polypeptide amino acid sequences and the nucleic acid sequences that code for these polypeptides. The databases come in a variety of different types that have advantages and disadvantages when viewed as the hypothesis for a polypeptide identification experiment.

While the “database entry” for an amino acid sequence may appear to be a simple text file to a user browsing for a particular polypeptide, many databases are organized into very flexible, complicated structures. The detailed implementation of the database on a particular system may be based on a collection of simple text files (a “flat-file” database), a collection of tables (a “relational” database), or it may be organized around concepts that stem from the idea of a protein, gene, or organism (an “object-oriented” database).

Protein mass data may be predicted from nucleic acid sequence databases. Alternatively, protein mass data may be obtained directly from protein sequence databases which contain a collection of amino acid sequences represented by a string of single-letter or three-letter codes for the residues in a polypeptide, starting at the N-terminus of the sequence. These codes may contain nonstandard characters to indicate ambiguity at a particular site (such as “B” indicating that the residue may be “D” (aspartic acid) or “N” (asparagine). The sequences typically have a unique number-letter combination associated with them that is used internally by the database to identify the sequence, usually referred to as the accession number for the sequence.

Databases may contain a combination of amino acid sequences, comments, literature references, and notes on known posttranslational modifications to the sequence. A database that contains these elements is referred to as “annotated.” Annotated databases are used if some functional or structural information is known about the mature protein, as opposed to a sequence that is known only from the translation of a stretch of nucleic acid sequence. Non-annotated databases only contain the sequence, an accession number, and a descriptive title.

The method for evaluating the performance of biopolymer identification algorithms includes the generation of signal and noise data sets.

In one embodiment of the invention the generation of signal data sets includes designating a biopolymer from a database as a signal biopolymer. The biopolymer chosen to be the signal biopolymer is a biopolymer that every algorithm, that is to be evaluated, can identify. Mass data obtained under a particular experimental condition is provided for the signal biopolymer.

Additionally, in this embodiment a biopolymer constituent part pool is provided which contains mass data of biopolymers from the database obtained under the same experimental condition used to generate the signal mass data. This mass data is selected randomly from the database. Accordingly, the pool can contain, but does not necessarily contain, the mass data of the signal biopolymer. The number of masses selected from the database to form the constituent part pool is in the range from one part to 100% of the total parts; preferably in the range from 50%-100%; and most preferably in the range from 90%-100%.

For example, theoretical protein sequences from NCBI's non-redundant protein sequence database can be cleaved (using trypsin cleavage rule) into peptides to form random monoisotopic peptide mass pools.

At least two and at most 10¹⁰ signal data sets are generated. Preferably 50 to 2000 signal data sets are generated. More preferably 200 to 1000 signal data sets are generated. It is preferable to have the same number of signal data sets as noise data sets. The signal data sets include mass data arbitrarily selected from the mass data generated for the signal biopolymer and include mass data selected from the pool. Arbitrary selection includes randomly selecting at least one mass from the signal biopolymer mass data and randomly selecting at least one mass from the pool. Arbitrary selection also includes selecting, by a nonrandom pattern, at least one mass from the signal biopolymer mass data and randomly selecting at least one mass from the pool. A nonrandom pattern includes any nonrandom pattern of selecting mass data. For example, a nonrandom pattern of selection includes selecting masses which are associated with fixed ordinal numbers, i.e., every fifth mass or every tenth mass of the signal mass data. The quantity of masses in each signal data set can range from about 1 to 50; more preferably from 2 to 40 and most preferably from 4to 30.

Accordingly, the signal data sets can be considered to be the signal protein with the addition of noise. This is done in order to degrade the quality, or strength, of the signal so that the sensitivity of different algorithms can be differentiated. The mass data of the signal data sets selected from the signal protein can be referred to as signal. The amount of signal in the signal data sets necessary to evaluate algorithms is the amount necessary to distinguish the performance of one algorithm from another algorithm. For instance, if the signal data sets contain too much signal, then virtually all algorithms tested may identify the correct biopolymer. In such a case the sensitivity, or performance, of the individual identification algorithms cannot be differentiated. Analogously, if the signal data sets contain too little signal, then the probability is low that any of the algorithms will identify the correct biopolymer thereby similarly not enabling the differentiation between algorithms. The amount of signal necessary to enable differentiation of algorithms depends on many factors. These factors include, for example, the number of masses in the data sets, the quality of the data in general and the molecular weight of the signal protein. The amount of signal in the signal data sets is increased with the number of masses in the signal data sets. The amount of signal in the signal data sets is decreased as the quality of the data decreases and the molecular weight of the signal protein increases. The amount of signal in each signal data set can range from at least one mass selected from the signal protein to all masses selected from the signal protein.

At least two and at most 10¹⁰ noise data sets are generated. Preferably 50 to 2000 noise data sets are generated. More preferably 200 to 1000 noise data sets are generated. It is preferable to have the same number of noise data sets as signal data sets. The noise data sets include the mass data selected from the constituent part pool. The mass data is selected from the pool arbitrarily. Arbitrary selection includes selection at random or selection by an arbitrary pattern. It is preferred to select the mass data at random. The number of masses in the noise data sets is preferably the number of total masses in the signal data sets or the number of masses in the signal data sets that are selected from the pool.

For example for proteins, each signal data set consists of a number of monoisotopic masses from a chosen protein sequence cleaved by a protease, for example, trypsin and a number of masses randomly selected from the peptide pool of the same species origin. Each noise data set has the same number of monoisotopic masses as the number of masses randomly selected from the peptide pool in the signal data set.

A search for each data set is conducted using biopolymer identification algorithms. A search includes comparing each data set with the database biopolymers and assigning identification search results to each data set. In evaluating the sensitivity of different algorithms the same search parameters are used for each algorithm. Search parameters include, for example, the database searched, the enzyme used to cleave the signal biopolymer and the database biopolymers, the mass of the signal biopolymer within a certain range, the tolerance, possible missed cleavage sites on the biopolymer, possible modifications in the signal biopolymer and the charge states of the signal biopolymer.

Examples of Key parameters used in searching for proteins include:

| Enzyme | Trypsin | Missed cleavage | 1 |
|---|---|---|---|
| Protein mass | 0-3000 kDa | Partial | Oxidation |
| range | | Modification | at Met |
| Tolerance | 50 ppm | Charge state | MH+ |

Identification search results are recorded for each of the data sets. Identification search results include a measure of the similarity between the generated data sets and each of the database biopolymers; and the database biopolymer candidates associated with each of the measures. The measure of the similarity can be a score. The scores for the top candidates are recorded. In some cases some algorithms may provide more than one candidate with the same score. If the signal protein is among these candidates, then the biopolymer identification can be classified as correct or as incorrect.

From the biopolymer identification search results obtained for each signal data set, a signal distribution is generated. The signal distribution is generated by determining the probability of obtaining each of the identification search results (scores) from the signal data set database search. From the biopolymer identification search results obtained for each noise data set, a noise distribution is generated. The noise distribution is generated by determining the probability of obtaining each of the identification search results (scores) from the noise data set database search.

In another embodiment of the method for evaluating the performance of biopolymer algorithms, data sets to be used in a search are generated using a different arbitrary method of generating data sets. This arbitrary method is accomplished by perturbation of mass data. In this embodiment a database biopolymer is designated as a test biopolymer and its mass data is provided. At least two masses are selected from the mass data of the test biopolymer to form a primary data set. The amount of mass data in the primary data set can range from about 1 to 50; more preferably from 2 to 40 and most preferably from 4 to 30. The mass data in the primary data set is perturbed to generate new data sets. At least two and at most 10¹⁰ data sets are generated; preferably 50 to 2000 data sets are generated; and more preferably 200 to 1000 data sets are generated. The mass data of the primary data set is perturbed. Perturbation is minor addition or subtraction to the individual masses in the primary data accomplished without changing the total number of masses in the data set. The value added to each individual mass is from 0 to 10,000 ppm; preferably 2 to 10 ppm; and most preferably from 4 to 50 ppm. The lower limit on the value depends on the instrument that was used to generate the mass data. Currently MALDI can reach ˜5 ppm. FTMS can reach a range of ·1to 3 ppm. added. Fifty to 100% of the masses are perturbed; more preferably 80%- 100% and most preferably 100% of the masses are perturbed.

As described in the previous method, a search for each of these data sets is conducted using biopolymer identification algorithms. Also as described in the previous method, identification search results are recorded for each of the data sets. The top candidate for each data set is compared with the test biopolymer. If the top candidate is the test biopolymer then the top candidate is designated as a signal; and at least one of the other candidates associated with the data set is designated as noise. If the top candidate is not the test biopolymer then at least one of the candidates associated with the data set is designated as noise.

From the biopolymer identification search results designated as a signal, a signal distribution is generated, as described above. From the biopolymer identification search results designated as noise, a noise distribution is generated, as described above. By generating separate distributions for signal and noise, correct identifications and random matches can be considered as from two different distributions.

In both embodiments of evaluating the performance of biopolymer identification algorithms, the noise and signal distributions are used to calculate at least one performance index. A performance index provides an objective standard by which to evaluate the quality, sensitivity or reliability of a biopolymer identification algorithm.

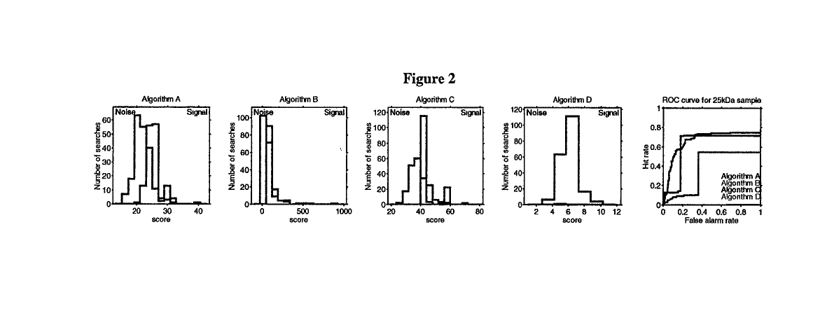

An example of a performance index is the index for separability and is designated as d′ (FIG. 2). This index is the distance between the mean of the noise distribution and the mean of the signal distribution. The value of d′ is a constant objective standard by which to evaluate an algorithm's sensitivity to differentiate noise from signal. The larger the value of d′ the better the two distributions are separated. Therefore, larger d′ are associated with more sensitive identification algorithms. When the two distributions can be approximated as normal distributions, d′ can be calculated by:

$${d}^{′} = \frac{{μ}_{s} - {μ}_{n}}{\sqrt{\frac{{σ}_{s} + {σ}_{n}}{2}}}$$

wherein μₛ and μₙ are the mean of the signal and noise distribution, respectively, and wherein σₛ and σₙ are the standard deviations of the signal and noise distributions, respectively.

In the above methods for evaluating the performance of algorithms, d′ is calculated from the noise and signal distributions generated by at least two different identification algorithms. The d′ calculated for each algorithm is compared to determine which algorithm has a better performance The algorithm that produces the greater d′ has the better performance.

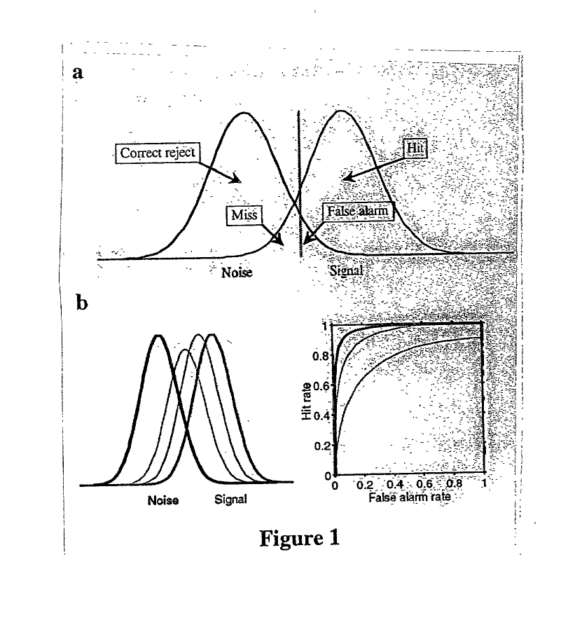

In order to describe another objective performance index, the “receiver operating characteristics (ROC)” curve, a subjective user selected decision criterion first needs to described. As seen from FIG. 1a, the decision criterion is a vertical line dividing the two distributions into four different regions. A user accepts identification results as correct which are to the right of the criterion.

Using Figure la as a model, one can see that as the criterion is moved to the left more of the signal distribution is to the right of the criterion. The signal distribution to the right of the criterion contains results that are hits. Hits are results which are from the signal population and a user decides are correct identifications. However, where the signal and noise distributions overlap on the right of the criterion, those results from the noise population are false alarms. They are false alarms since they come from the noise distribution and the user has defined these results as being correct identifications.

To the left of the criterion there is a region of correct rejections. Correct rejections are results which have been classified as random identifications by the user and are actually random identifications. However, also to the left of the criterion there is a region of misses. Results designated as misses are those which the user classifies as random identifications but are actually correct identifications.

As the user moves the criterion to the left the number of misses decrease. At the same time, however, the probability of false alarms increases. Moving the criterion to the left is considered to be more liberal.

As the user moves the criterion to the right, on the other hand, the probability of false alarms decreases. At the same time, however, the probability of misses increases. Moving the criterion to the right is considered to be more stringent.

To eliminate the subjectivity inherent in choosing a criterion, a ROC curve is generated. The ROC curve is the plot of the hit and false-alarm probabilities at each possible value on the score axis. (See Figure lb.) In a ROC curve, as shown in Figure lb, points toward the upper left corner correlates with a better separation of the signal and noise populations. The area (A′) under such a ROC curve is a performance index of an algorithm. The index measures the sensitivity of the algorithm. Another index is the maximum hit rate (Hit_{MAX}) which is the point on the ROC curve at the false alarm rate of one. These indices are independent of decision criterion or choice preference (stringent, moderate or liberal). The larger the A′ value the better the algorithm distinguishes the correct candidate from random matches, i.e., the better the performance of the algorithm. Performance of an algorithm is directly proportional to A′.

The ROC curve can be generated in a number of ways. As illustrated above, A′ can be the area under the curve of the plot of the hit rate (probability) on the y-axis and the false alarm rate (probability) on the x-axis. The ROC curve can also be plotted with the hit rate (probability) on the x-axis and the false alarm rate (probability) on the y-axis. Additionally the axes can have a reverse scale; that is, the units on the axes can be decreasing instead of increasing as going farther away from the origin. In these cases, the area A′ defined by the ROC curve should be calculated appropriately, as would be known by one skilled in the art. Additionally, A′ can be calculated as the complement of the area defined by the curve. Additionally, the probability of hit/false alarm can be expressed in Z score. For example, for normal distributions, the probability is the integration of the area under the probability density function from negative infinity to Z. For instance, p=0.95 corresponds to Z=1.65. In such a case, the ROC would be a straight line when the x and y axes are using linear Z.

In the above methods for evaluating the performance of algorithms, ROC curves can be plotted and A's calculated for each algorithm. The A's are compared for at least two biopolymer identification algorithms. The algorithm associated with the greater A′ value is the algorithm with the better performance.

The methods described above for evaluating the performance of algorithms include evaluating the performance of different versions of the same algorithm. An algorithm can be evaluated using particular search parameters and data. Then a second algorithm which is a modified version of the first algorithm can be evaluated using the same search parameters and same data. Modifications of the algorithm can include any variables, assumptions and calculations that an algorithm uses to generate a score. The algorithm which demonstrates the greater indices is the better, or improved, version of the algorithm. Modifications and evaluations can be repeated numerous times to fine tune an identification algorithm.

In another embodiment of the present invention a method for evaluating the reliability of a biopolymer identification result for an unknown biopolymer is provided. The method includes generating noise and signal distributions. The noise distribution includes identification results obtained from the search of a database for arbitrarily generated sets of database mass data. The signal distribution includes identification results obtained from the search of a database for arbitrarily generated sets of database mass data that includes mass data of the unknown biopolymer. Performance indices are calculated from the distributions by which the reliability of the identification result can be evaluated.

In one embodiment of the method for evaluating a biopolymer identification result, the method includes providing mass data of a candidate associated with a biopolymer identification result obtained from searching a biopolymer data base for an unknown biopolymer with a particular identification algorithm. This mass data is obtained under particular experimental conditions.

As described above for the method to evaluate algorithms, a constituent part pool is provided. The pool includes mass data of database biopolymers obtained under the same experimental condition as used to generate the mass data of the unknown biopolymer. The database used is selected by the user. As described above, noise data sets are generated including the mass data from the pool.

Signal data sets are generated. These signal data sets include mass data of the candidate associated with the biopolymer identification result of the unknown biopolymer and mass data from the pool. (In comparison to the method for evaluating algorithms described above, the mass data of the unknown protein is used instead of the mass data of the signal protein to generate these signal data sets.)

As described above, a search of the database for each data set is conducted using the identification algorithm to obtain at least one identification search result for each of the data sets.

As described above, a signal distribution of the identification search results obtained from the search of the signal data sets is generated. As described above, a noise distribution of the identification search results obtained from the search of the noise data sets is generated.

As described above, at least one performance index is generated from the distributions. The value of d′ describes how well the signal and noise was separated when producing the identification result. The value of A′ provides the probability of hits and false alarms obtained for the identification result. These indices reflect of the reliability of the protein identification result.

In another embodiment of the method for evaluating a biopolymer identification result, data sets are generated by using perturbation. A search is performed for an unknown biopolymer using a particular identification algorithm to obtain biopolymer identification results. Mass data of the top candidate is designated as the original candidate. This mass data is obtained under particular experimental conditions.

As described above, mass data is selected from the mass data of the original candidate to form a primary data set. The mass data selected to form the primary data set is preferably all the mass data of the original candidate.

As described above, at least two additional data sets are generated by perturbing the mass data of the primary data set.

As described above, a search of the data base is conducted, using particular search parameters, for each data set using the protein identification algorithm to obtain at least one candidate, designated as a data set candidate, for each of the data sets.

As described above, it is determined whether a top data set candidate is the original candidate. If the top data set candidate is the original candidate then the top data set candidate is designated as a signal and at least one of the other data set candidates are designated as noise. If the top data set candidate is not the original candidate then at least one of the other data set candidates are designated as noise.

As described above noise and signal distributions are generated.

As described above, at least one performance index is generated from the distributions. The values of d′ and A′ describe how well the signal and noise have been separated when producing the identification result. The probability of hits for a given false alarm probability for the biopolymer identification result is determined from the distributions. Accordingly, these indices reflect of the reliability of the protein identification result.

The method for evaluating the reliability of an identification result can further include optimizing the search parameters for a protein identification result. Search parameters are user-selected parameters. Based on knowledge that a user has about a particular unknown biopolymer, a user can constrain the search of a data base taking these factors into account.

The evaluation of the reliability of an identification result is repeated for different sets of search parameters for the same unknown biopolymer and algorithm. At least one performance index is calculated from the distributions for each set of search parameters. The performance indices associated with each set of parameters are compared. It is determined which set of search parameters provides the best performance indices, thereby optimizing the protein identification result.

Examples of search parameters include the database searched, the species of the unknown, search for mixtures of biopolymers, a constraint on the mass range to be searched, information on the pI range for proteins, number of missed cleavage sites, enzyme cleavage rules, complete possible modifications of the unknown, partial modifications of the unknown, tolerance type (absolute/relative) and tolerance value.

The observed molecular mass or the observed isoelectric point of a protein can be used in combination with the measured masses of peptides generated by proteolysis to constrain the search for a polypeptide. In particular, the comparison between the theoretical mass data of the database proteins and the mass data of the unknown protein may be constrained to only those proteins of the database which are within a chosen mass range. The chosen mass range may be constrained to within a certain percentage of the mass of the unknown protein. However, since proteins may be degraded, such a constraint may possibly increase misses. Peptide mass range may also be restricted. The preferred range is 500Da to 5000Da.

Similarly, the comparison between the theoretical mass data of the database proteins and the mass data of the unknown protein may be constrained to only those proteins of the database which are within a chosen isoelectric point range. The isoelectric point (pI) of a protein is the pH at which its net charge is zero. The chosen isoelectric point range is preferably within 50% of the isoelectric point of the unknown protein, more preferably within 35%, most preferably within 25%. However, since proteins may be degraded, such a constraint may possibly increase misses.

Using the observed molecular mass or isoelectric point of a polypeptide to constrain a search must be done carefully. When nonannotated nucleotide sequence databases are used (such as TREMBL or GENPEPT), subsequent processing can greatly alter the pI or molecular mass of a protein, so much so that no identification can be made. For example, the small, highly conserved protein ubiquitin (SWISSPROT accession number P02248) has a molecular mass of 8.6 kD, which is the mass that would be measured by a mass spectrometer or a gel. A simple keyword search of the translated-nucleotide database GENPEPT results in several sequences for the same protein [accession numbers M26880 (77 kD), U49869 (25.8 kD) and X63237 (17.9 kD)]. None of these nucleotide-translated sequences give the correct molecular mass or pI, so using those parameters to limit a search would result in missing the database sequence altogether. Only annotated databases that fully outline known modifications should be used when the properties of the mature protein are being used to constrain a search.

Biopolymers may undergo common modifications in their structure. The mass data that are generated from a biopolymer database may include mass data representing biopolymers with common modifications.

Examples of such modifications are posttranslational modifications of proteins. The modification state of a protein is usually not known in detail. In database searches, it can be useful to assume that some common modifications might be present. This is achieved by comparing the measured peptides masses of the unknown protein with both the masses of the unmodified and modified peptides in the database.

Examples of posttranslational modifications include glycosylation and the oxidation of the amino acid methionine. Another example is the phosphorylation of the amino acids serine, threonine, and tyrosine. Phosphorylation is often used to activate or deactivate proteins and the phosphorylation state of an experimentally observed protein depends on may factors including the phase of the cell cycle and environmental factors.

Optionally, further information of the unknown protein's sequence is obtained by generating fragment mass data. Fragment mass data for a peptide can be generated in any manner which provides fragment mass data within a certain accuracy. Experimental conditions include the type of energy used to generate the fragment mass data. Vibrational excitation energy can be used. The vibrational excitation may be generated by collisions of the peptide with electrons, photons, gas molecules or a surface. Electronic excitation can be used. The electronic excitation may be generated by collisions of the peptide with electrons, photons, gas molecules (e.g. argon) or a surface.

In another example, the experimental fragment mass spectrum of a peptide from an enzymatically digested unknown protein is compared with the theoretical masses calculated by applying the rules for the specificity of the enzyme, and the rules for the fragmentation as known to those of ordinary skill in the art, to the amino acid sequence of a database protein. For example, the software tool PepFrag (ProteoMetrics) allows for searching protein or nucleotide sequence databases using a combination of mass spectra data and fragmentation mass spectra data.

For example, fragment mass data for the purposes of this invention can be generated by using multidimensional mass spectrometry (MS/MS), also known as tandem mass spectrometry. A number of types of mass spectrometers can be used including a triple-quadruple mass spectrometer, a Fourier-transform cyclotron resonance mass spectrometer, a tandem time-of-flight mass spectrometer, and a quadruple ion trap mass spectrometer. A single peptide from a protein digest is subjected to MS/MS measurement and the observed pattern of fragment ions is compared to the patterns of fragment ions predicted from database sequences.

In one embodiment the present invention provides a means for evaluating the performance of biopolymer identification algorithms. The means is any means by which the performance can be evaluated. For example, the means includes a computer and/or mass spectra, as would be recognized by a person skilled in the art. Included in the means is a means for generating noise and signal distributions. The noise distribution comprises identification results obtained from the search of a database for arbitrarily generated sets of database mass data. The signal distribution comprises identification results obtained from the search of a database for arbitrarily generated sets of database mass data which comprises mass data of a particular biopolymer of the database designated as a signal biopolymer. Also included is a means for calculating a performance index from the distributions which evaluates the performance of the algorithm.

In one embodiment the present invention provides a means for evaluating the reliability of a biopolymer identification result. The means is any means by which the performance can be evaluated. For example, the means includes a computer or mass spectra, as would be recognized by a person skilled in the art. Included in the means is a means for generating noise and signal distributions, wherein the noise distribution comprises identification results obtained from the search of a database for arbitrarily generated sets of database mass data and wherein the signal distribution comprises identification results obtained from the search of the database for arbitrarily generated sets of database mass data and mass data of the unknown biopolymer; and a means for calculating a performance index from the distributions which is a function of the reliability of a biopolymer identification result.

In one embodiment the present invention provides a computer program product including a computer usable medium having computer readable program code meals embodied in said medium for evaluating the performance of biopolymer identification algorithms. The computer program product includes a computer readable program code means for causing a computer to generate noise and signal distributions, wherein the noise distribution comprises identification results obtained from the search of a database for arbitrarily generated sets of database mass data and wherein the signal distribution comprises identification results obtained from the search of a database for arbitrarily generated sets of database mass data which comprises mass data of a particular biopolymer of the database designated as a signal biopolymer; and a computer readable program code means for causing a computer to generate a performance index from the distributions which evaluates the performance of the algorithm.

In one embodiment the present invention provides a computer program product including a computer usable medium having computer readable program code means embodied in said medium for a means for evaluating the reliability of a biopolymer identification result. The computer program product includes a computer readable program code means for causing a computer to generate noise and signal distributions, wherein the noise distribution comprises identification results obtained from the search of a database for arbitrarily generated sets of database mass data and wherein the signal distribution comprises identification results obtained from the search of the database for arbitrarily generated sets of database mass data and mass data of the unknown biopolymer; and a computer readable program code means for causing a computer to calculate a performance index from the distributions which is a function of the reliability of a biopolymer identification result.

### EXAMPLES

### Method Using Replacement

Peptide pool: Theoretical protein sequences from NCBI's non-redundant protein sequence database were cleaved (using the trypsin cleavage rules) into peptides to form a random peptide monoisotopic mass pool.

Signal data sets: Each signal data set includes a number of monoisotopic masses from a chosen protein sequence cleaved by trypsin and a number of masses randomly selected from the peptide pool of the same species. These randomly selected peptides were included to simulate real data—i.e. make the signal weaker.

Noise data set: Each noise data set has the same number of monoisotopic masses as the corresponding signal data set. These masses are randomly selected from the same peptide pool used for the signal data set.

Key parameters: Search parameters were selected to accommodate different search programs and the same parameters were used throughout all searches.

| Enzyme | Trypsin | Missed cleavage | 1 |
|---|---|---|---|
| Protein mass | 0-3000 kDa | Partial | Oxidation |
| range | | Modification | at Met |
| Tolerance | 50 ppm | Charge state | MH+ |

Simulation steps

1. Parameters and mass data were sent to different search programs

2. The scores for the top candidates were recorded

3. Signal and noise distributions were estimated by plotting the scores for the signal and noise data sets

4. ROC curves were plotted and A's and Hitₘₐₓ were calculated for each search program.

### Results

Four different algorithms were tested in this study. They are “Bayesian” (ProFound), “MOWSE (modified)” (MS-Fit), “MOWSE (probability based)” (Mascot) and “Number of Matches” (MS-Fit). They are denoted as algorithms A, B, C, and D, respectively. Four proteins (25 kDa from yeast, 50 kDa from mouse, 100 kDa from human, and 200 kDa from algorithm C. elegans) were chosen for this test. 200 signal data sets and 200 noise data sets were used for each sample protein.

FIG. 2 shows the results for the 25 kDa protein. Each signal data set in this experiment contains 4 signal peptides and 16 random peptides. Note algorithm D performed very poorly in this comparison.

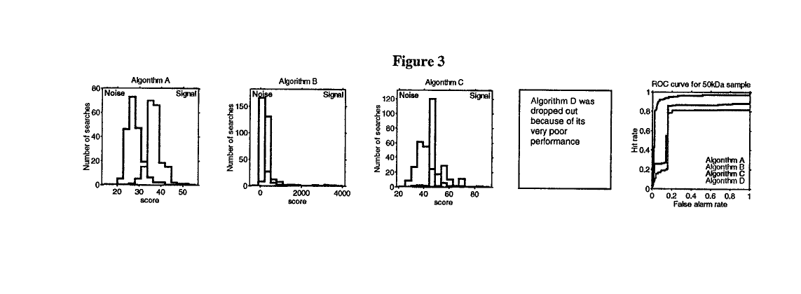

FIG. 3 shows the results for a 50 kDa protein. Each signal data set in this experiment contains 6 signal peptides and 24 random peptides.

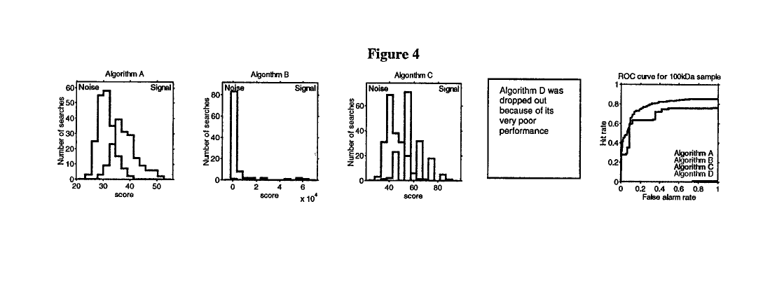

FIG. 4 shows the results for a 100 kDa protein. Each signal data set in this experiment contains 8 signal peptides and 24 random peptides. Note algorithm B performed very poorly in this comparison.

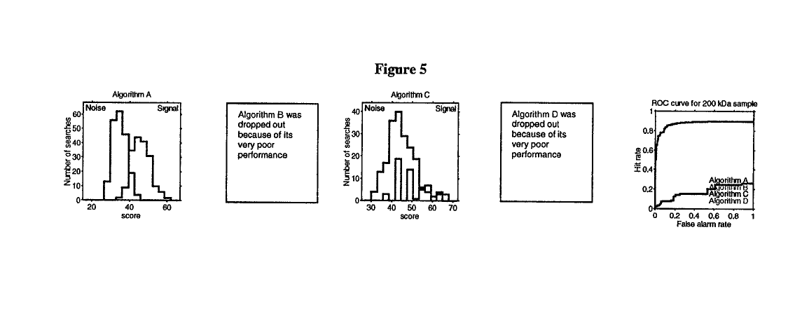

FIG. 5 shows the results for a 200 kDa protein. Each signal data set in this experiment contains 10 signal peptides and 30 random peptides.

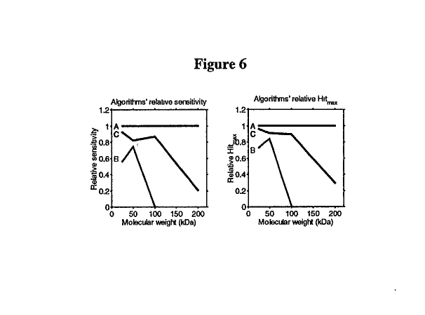

FIG. 6 shows a) the relative sensitivity (A′/ A′ₘₐₓ) and b) the relative maximum hit rate (Hitₘₐₓ/max(Hitₘₐₓ)) of the three algorithms (A, B, C). Algorithm A outperforms the other algorithms throughout the molecular weight range tested. The optimum performance is around molecular weight 50 kDa for algorithm B and less than 100 kDa for algorithm C. The optimum performance molecular weight range for Algorithm B is the narrowest.

### Conclusions

1. Signal detection theory can be used for evaluating the performance of database search algorithms for protein identification objectively.

2. Sensitivity differences exist between different search algorithms. Algorithm A showed the best sensitivity for our test data, followed by algorithm C and B. Algorithm D showed the worst performance.

### Method Using Perturbation

Database Search

ProFound available at (http://www.proteometrics.com/) tailored for the present study is used to perform database searching to output the raw scores —e based logarithm of the likelihood —for the candidate proteins.

Signal and Noise Population Estimation

Given one set of experimental mass data, the IntelliDuo© algorithm is used to generate the estimated populations for the signal and noise. The signal and noise populations respectively reflect the score distributions for the correctly identified protein and for randomly matched protein candidates when many sets of experimental data (obtained under the same experimental conditions) are used in the search.

Experimental Data Sets

To generate the experimental signal and noise populations, 100 MALDI mass spectra for tryptic digests of a 36 kDa yeast protein (GPD3) were acquired under the same experimental condition on a modified Sciex QqTOF. Each data set contains masses of 16 chosen peaks having the strongest intensities.

ROC Curve and A′

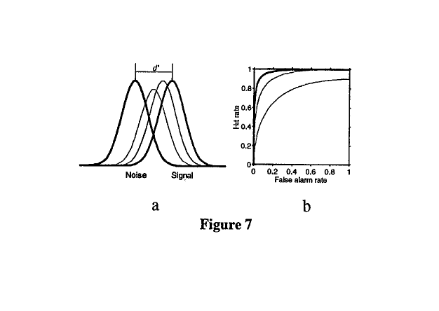

For given signal and noise populations, one form of ROC (Receiver Operating Characteristic) curves is a plot of hit rate against false-alarm rate for every possible value on the score axis (FIG. 7a). It describes the relationship between the hit rate and false-alarm rate (FIG. 7b). A ROC curve toward the upper left corner correlates with a better separation between the two populations. A′ is the area under the ROC curve, which is also a measure for the separation. Larger A′ is better.

Index of Separability

The index of separability, d′, represents the separation between the signal and noise populations (FIG. 7). The larger the d′ value, the better separation of the two distributions. When the two distributions can be approximated by normal distributions, d′ is given by:

$${d}^{′} = \frac{{μ}_{s} - {μ}_{n}}{\sqrt{\frac{{σ}_{s}^{2} + {σ}_{n}^{2}}{2}}}$$

where μ and σ are the mean and standard deviation of a population respectively. The subscripts s and n signify the signal and noise populations respectively.

### Results

Signal and Noise Population and Their Estimates

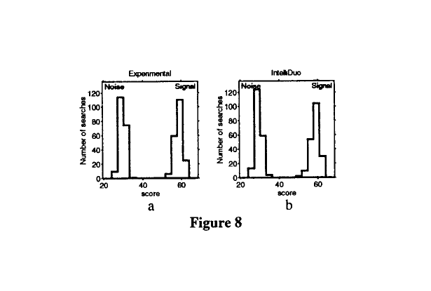

FIG. 8a and 8b show the signal and noise populations for the 100 experimental data sets and its estimates using the IntelliDuo© algorithm from one set of experimental data. Ten out of sixteen masses matched GPD3 while six did not match. The similarity between the two sets of populations shows that the IntelliDuo© algorithm can be used to estimate the signal and noise populations.

| | Signal population | | Noise population | |
|---|---|---|---|---|
| | Experimental | Estimated | Experimental | Estimated |
| Mean | 58.7 | 58.5 | 29.5 | 29.4 |
| S.D. | 2.2 | 2.3 | 1.7 | 1.7 |
| d′ | 15.0 | 14.4 | | |

Examples of Estimated Signal and Noise Populations

As examples of population estimates generated using the IntelliDuo © algorithm, we present our observations on the relationships between signal and noise populations as a function of data set size, modification and species specification.

Size of the Data Set

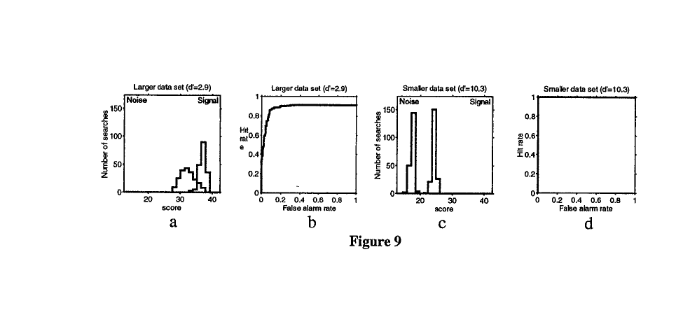

The signal and noise populations for two experimental data sets of different size are estimated. In both searches (using the experimental data sets) ProFound places the correct candidate (human CFTR, 168 kDa) at the 1^{st} place (probabilities˜1 and Z˜1) with similar discrimination against randomly matched candidates (probabilities˜10⁻³). FIG. 9a shows population estimates for one set of experimental data with six matches out of total 43 masses. The two populations overlap due to the low signal (six matches) to noise (37 mismatches) ratio. In the ROC curve shown in FIG. 9b, it can be read out that the hit rate at 5% false-alarm rate is 75% and the maximum hit rate is 91% (at false-alarm rate of 100%). FIG. 9c is for a subset of the above data with 4 matches out of total 9 masses and it shows a larger separation than FIG. 9a. This observation is consistent with our experience that, for small data sets, the ProFound often finds the correct candidate even though its discrimination against the randomly matched candidates is small. Thus the signal detection theory analysis provides a normalized measure with respect to different sizes of data sets. FIG. 9d is the ROC curve for the two populations shown in FIG. 9c where at any false-alarm rate; the hit rate is 100%.

Modification

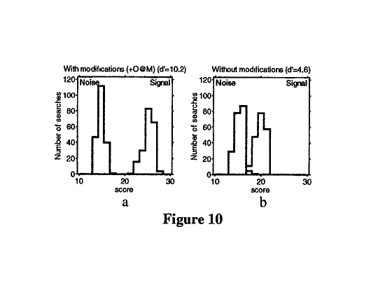

The effect of including partial oxidation on methionine residues is shown in FIG. 10aand 10b. FIG. 10a shows the population estimates from a total of eight experimental masses where four match with the unmodified authentic protein (yeast Nup170). In FIG. 10b where partial oxidation is included in the search, an additional match shifted the signal population to a higher score while the noise population changed very little. Hence, the separation between the signal and noise populations is increased. However, caution should be exercised when using partial phosphorylation. Although the signal population could shift to higher scores, the high abundance of S/T/Y residues in the database would at the same time shift the noise population to higher scores. Whether the population separation increases depends on the extent of the shifts of both populations.

Species

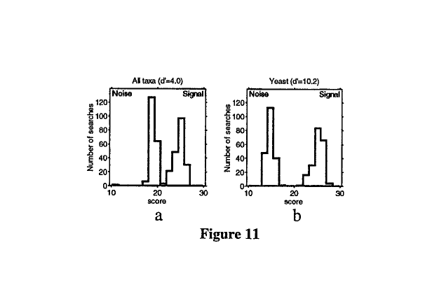

The species of origin of a sample protein is known to be a useful constraint for protein identification. The population estimates for a set of experimental data (from yeast Nup 170) is shown in FIG. 11a where “all-taxa” is used in database searches. When the yeast species information is used in searches, the signal population does not change, while the noise population shifts to a lower score as shown in FIG. 11b. Thus, the separation between the two populations increases.

### Conclusions

Signal detection theory provides proper framework for an objective evaluation of database search result.

We claim:

1. A method for evaluating the performance of biopolymer identification algorithms, the method comprising:
a) generating noise and signal distributions, wherein the noise distribution comprises identification results obtained from the search of a database for arbitrarily generated sets of database mass data and wherein the signal distribution comprises identification results obtained from the search of a database for arbitrarily generated sets of database mass data which comprises mass data of a particular biopolymer of the database designated as a signal biopolymer; and
b) calculating a performance index from the distributions which evaluates the performance of the algorithm.

2. The method of claim 1 wherein the biopolymer is a protein.

3. The method of claim 1 wherein the biopolymer is a nucleic acid molecule.

4. The method of claim 1 wherein the biopolymer is a polysaccharide.

5. A method according to claim 1 for evaluating the performance of biopolymer identification algorithms, the method comprising:
a) providing a constituent part pool comprising mass data of biopolymers from a database obtained under an experimental condition;
b) designating a biopolymer from the database as a signal biopolymer and providing mass data of the signal biopolymer obtained under the experimental condition;
c) generating at least two signal data sets comprising mass data of the signal biopolymer and mass data from the constituent part pool;
d) generating at least two noise data sets comprising the mass data from the constituent part pool;
e) conducting a search of the database using a biopolymer identification algorithm to obtain at least one biopolymer identification search result for each of the signal data sets and for each of the noise data sets;
f) generating a signal distribution of the identification search results obtained from the search of the signal data sets;
g) generating a noise distribution of the identification search results obtained from the search of the noise data sets;
h) calculating at least one performance index from the distributions;
i) repeating steps (e) to (h) with a second biopolymer identification algorithm; and
j) comparing the performance index of the biopolymer identification algorithm and the second biopolymer identification algorithm to determine which algorithm has a better performance.

6. The method according to claim 5 wherein a noise data set is generated by randomly selecting at least two masses from the constituent part pool.

7. The method according to claim 5 wherein generating a signal data set comprises:
a) randomly selecting at least one mass from the signal biopolymer mass data; and
b) randomly selecting at least one mass from the constituent part pool; thereby generating a signal data set.

8. The method according to claim 5 wherein generating a signal data set comprises:
a) selecting at least one mass from the signal biopolymer mass data by a nonrandom pattern; and
b) randomly selecting at least one mass from the constituent part pool; thereby generating a noise data set.

9. The method according to claim 5 wherein the signal data sets and the noise data sets have the same number of masses.

10. The method according to claim 5 wherein the noise data sets have the same number of masses as the number of masses in the signal data set selected from the constituent part pool.

11. The method according to claim 5 wherein the performance index is the distance (d′) between the mean of the noise distribution and the mean of the signal distribution and wherein the performance is directly proportional to d′.

12. The method according to claim 5 wherein the performance index is the area (A′) defined by a curve of the plot of probability of hits on one axis and the probability of false alarms on the second axis wherein the performance is directly proportional to A′.

13. The method according to claim 5 wherein the second algorithm is a modified version of the algorithm and wherein the method is used to improve the algorithm.

14. A method according to claim 1 for evaluating the performance of biopolymer identification algorithms, the method comprising:
a) providing mass data for at least one biopolymer from a database and designating the biopolymer as a test biopolymer;
b) selecting at least two masses from the mass data to form a primary data set;
c) generating a sufficient number of additional data sets by perturbing the mass data of the primary data set;
d) conducting a search of the database for each data set using a biopolymer identification algorithm to obtain at least one biopolymer identification search result for each of the data sets;
e) determining whether a top candidate obtained in the search for each data set is the test biopolymer;
f) if the top candidate is the test biopolymer then designating the top candidate as a signal and designating at least one of the other candidates as noise;
g) if the top candidate is not the test biopolymer designating at least one of the candidates as a noise;
h) generating a distribution of the signal;
i) generating a distribution of the noise;
j) calculating at least one performance index from the distributions;
k) repeating steps (d) to (j) with a second biopolymer identification algorithm; and
l) comparing the performance index of the biopolymer identification algorithm and the second biopolymer identification algorithm to determine which algorithm has a better performance.

15. The method according to claim 14 wherein the performance index is the distance (d′) between the mean of the noise distribution and the mean of the signal distribution and wherein the performance is directly proportional to d′.

16. The method according to claim 14 wherein the performance index is the area (A′) defined by a curve of the plot of probability of hits on one axis and the probability of false alarms on the second axis wherein the performance is directly proportional to A′.

17. The method according to claim 14 wherein the second algorithm is a modified version of the algorithm and wherein the method is used to improve the algorithm.

18. A method for evaluating the reliability of a biopolymer identification result, the method comprising:
a) generating noise and signal distributions, wherein the noise distribution comprises identification results obtained from the search of a database for arbitrarily generated sets of database mass data and wherein the signal distribution comprises identification results obtained from the search of the database for arbitrarily generated sets of database mass data and mass data of the unknown biopolymer; and
b) calculating a performance index from the distributions which is a function of the reliability of a biopolymer identification result.

19. The method of claim 18 wherein the biopolymer is a protein.

20. The method of claim 18 wherein the biopolymer is a nucleic acid molecule.

21. The method of claim 18 wherein the biopolymer is a polysaccharide.

22. A method according to claim 18, the method comprising:
a) providing mass data of a candidate, designated as the original candidate, associated with a biopolymer identification result obtained from searching a biopolymer data base for an unknown biopolymer with a biopolymer identification algorithm with selected search parameters;
b) selecting mass data from step (a) to form a primary data set;
c) generating at least two additional data sets by perturbing the mass data of the primary data set;
d) conducting a search of the data base, using the search parameters, for each data set using the biopolymer identification algorithm to obtain at least one candidate, designated as a data set candidate, for each of the data sets;
e) determining whether a top data set candidate is the original candidate,
f) if the top data set candidate is the original candidate then designating the top data set candidate as a signal and designating at least one of the other data set candidates as noise;
g) if the top data set candidate is the original candidate then designating at least one of the candidates as a noise;
h) generating a distribution of the signal;
i) generating a distribution of the noise; and
j) calculating at least one performance index from the distributions; and
k) determining from the performance index the reliability of the identification result.

23. The method according to claim 22 wherein the probability of hits for a given false alarm probability for the biopolymer identification result is determined from the distributions.

24. A method according to claim 22 further comprising optimizing a biopolymer identification result for the unknown biopolymer:
a) repeating the method for different sets of search parameters of the biopolymer identification algorithm; and
b) calculating at least one performance index from the distributions for each set of parameters;
c) comparing the performance index associated with each set of search parameters;
d) determining the search parameters which provide the best performance index, thereby optimizing the biopolymer identification result for the unknown biopolymer.

25. A method according to claim 18 for evaluating a biopolymer identification result, the method comprising:
a) providing mass data obtained under a experimental condition of a candidate associated with a biopolymer identification result obtained from searching a biopolymer data base for an unknown biopolymer with a biopolymer identification algorithm;
b) providing a constituent part pool comprising mass data of biopolymers from the database obtained under the experimental condition;
c) generating at least two signal data sets comprising mass data of the candidate associated with a biopolymer identification result of the unknown biopolymer and mass data from the constituent part pool;
d) generating at least two noise data sets comprising the mass data from the constituent part pool;
e) conducting a search of the database using the biopolymer identification algorithm to obtain at least one biopolymer identification search result for each of the signal data sets and for each noise data sets;
f) generating a signal distribution of the identification search results obtained from the search of the signal data sets;
g) generating a noise distribution of the identification search results obtained from the search of the noise data sets;
h) calculating at least one performance index from the distributions; and
i) determining from the performance index the reliability of the identification result.

26. The method according to claim 25 wherein the probability of hits for a given false alarm probability for the biopolymer identification result is determined from the distributions.

27. A method according to claim 25 further comprising optimizing a biopolymer identification result for the unknown biopolymer:
a) repeating the method for different sets of search parameters of the biopolymer identification algorithm; and
b) calculating at least one performance index from the distributions for each set of parameters;
c) comparing the performance index associated with each set of search parameters;
d) determining the search parameters which provide the best performance index, thereby optimizing the biopolymer identification result for the unknown biopolymer.

28. A means for evaluating the performance of biopolymer identification algorithms comprising:
a) a means for generating noise and signal distributions, wherein the noise distribution comprises identification results obtained from the search of a database for arbitrarily generated sets of database mass data and wherein the signal distribution comprises identification results obtained from the search of a database for arbitrarily generated sets of database mass data which comprises mass data of a particular biopolymer of the database designated as a signal biopolymer; and
b) a means for calculating a performance index from the distributions which evaluates the performance of the algorithm.

29. A means for evaluating the reliability of a biopolymer identification result comprising:
a) a means for generating noise and signal distributions, wherein the noise distribution comprises identification results obtained from the search of a database for arbitrarily generated sets of database mass data and wherein the signal distribution comprises identification results obtained from the search of the database for arbitrarily generated sets of database mass data and mass data of the unknown biopolymer; and
b) a means for calculating a performance index from the distributions which is a function of the reliability of a biopolymer identification result.

30. A computer program product comprising:
a computer usable medium having computer readable program code means embodied in said medium for evaluating the performance of biopolymer identification algorithms, said computer program product including:
a) a computer readable program code means for causing a computer to generate noise and signal distributions, wherein the noise distribution comprises identification results obtained from the search of a database for arbitrarily generated sets of database mass data and wherein the signal distribution comprises identification results obtained from the search of a database for arbitrarily generated sets of database mass data which comprises mass data of a particular biopolymer of the database designated as a signal biopolymer; and
b) a computer readable program code means for causing a computer to generate
a performance index from the distributions which evaluates the performance of the algorithm.

31. A computer program product comprising:
a computer usable medium having computer readable program code means embodied in said medium for a means for evaluating the reliability of a biopolymer identification result, said computer program product including:
a) a computer readable program code means for causing a computer to generate noise and signal distributions, wherein the noise distribution comprises identification results obtained from the search of a database for arbitrarily generated sets of database mass data and wherein the signal distribution comprises identification results obtained from the search of the database for arbitrarily generated sets of database mass data and mass data of the unknown biopolymer; and
b) a computer readable program code means for causing a computer to calculate a performance index from the distributions which is a function of the reliability of a biopolymer identification result.

32. The method according to claim 1 wherein the mass data is fragment mass data.

33. The method according to claim 18 wherein the mass data is fragment mass data.

34. The method according to claim 22 wherein all the mass data of the original candidate is selected to form the primary data set.
